(19) United States
(12) Patent Application Publication
(10) Pub. No.: US 2011/0171607 A1
(43) Pub. Date: Jul. 14, 2011

(54) BIOPULP

(75) Inventors: Jeremy J. Mao, Closter, NJ (US); Eduardo K. Moioli, hicago, IL (US); Jin Young Kim, San Antonio, TX (US)

(73) Assignee: THE TRUSTEES OF COLUMBIA UNIVERSITY IN THE CITY OF NEW YORK, New York, NY (US)

(21) Appl. No.: 12/739,902

(22) PCT Filed: Oct. 23, 2008
(86) PCT No.: PCT/US08/81011

§ 371 Date: Oct. 20, 2010

Related U.S. Application Data
(60) Provisional application No. 60/982,671, filed on Oct. 25, 2007.
(60) Provisional application No. 61/041,681, filed on Apr. 2, 2008.

(51) Int. Cl. A61C 5/02; A61K 38/18; A61P 25/00; A61P 19/08

(52) U.S. Cl. 433/224; 514/8.4; 514/8.8; 514/8.1; 514/9.1

(57) ABSTRACT

Provided are methods for performing a dental, endodontic or root canal procedure on a mammalian tooth in need thereof. Also provided are matrices, materials or scaffolds suitable for insertion into a tooth pulp chamber. Additionally provided are uses of any of the above matrix, material or scaffolds in a dental, endodontic or root canal procedure. Further provided are uses of any of the above matrices, materials or scaffolds for the manufacture of a medicament for a dental, endodontic or root canal procedure.

### CROSS-REFERENCE TO RELATED APPLICATIONS

This application claims the benefit of U.S. Provisional Application No. 61/041,681, filed Apr. 2, 2008, and U.S. Provisional Application No. 60/982,671, filed Oct. 25, 2007. Both applications are herein incorporated by reference in their entirety.

### STATEMENT REGARDING FEDERALLY SPONSORED RESEARCH OR DEVELOPMENT

This invention was made with government support under Grant No. R01DE15391 awarded by The National Institutes of Health. The government has certain rights in the invention.

### BACKGROUND

The present application generally relates to dental treatments and compositions.

The tooth is biologically viable largely because of the tooth pulp. Currently, diseased, missing or traumatized dental pulp is treated by capping or replacement with inert synthetic materials. The most common filling material is gutta-percha, a thermoplastic polymer of isoprene. After removal of the native tooth pulp that has been diseased, is missing or is traumatized, gutta-percha is melted and injected to fill the root canal. Although endodontic or root canal treatment has been the conventional state of art of contemporary dentistry, it has several deficiencies that negatively affect the quality of life of the patient (Salvi et al. 2007). First, root canal-treated teeth tend to be brittle, and susceptible to fracture. Second, discoloration frequently takes place following root canal treatment. Patients whose root canal treated teeth have undergone discoloration often require additional and costly cosmetic dental procedures. Third, diseased, missing or infected tooth pulp of deciduous (baby) teeth often lacks treatment options and is frequently not suitable for root canal treatment. Pulp necrosis happens in 85-96% of the avulsed teeth and in 70-100% of the intruded teeth. Untreated or poorly managed dental infections may be the causes of systemic infections (Shay 2002; Brennan et al. 2007).

Ideally, an improved treatment for teeth having diseased, missing or traumatized pulp causes the restoration of biologically vital tissue. Tissue engineering techniques have been used in the development of methods and compositions for restoring craniofacial tissues and bone. See, e.g., Alhadlaq and Mao, 2003; Edwards and Mason, 2006; Fong et al., 2005; Goldberg and Smith, 2004; Hong and Mao, 2004; Lovschall et al., 2001; Mao et al., 2006; Mathieu et al., 2006; Murray et al., 2002; Murray et al., 2007; Nakashima and Alamine, 2005; Nakashima and Reddi, 2003; Stosich and Mao, 2007; Young et al., 2002; U.S. Pat. No. 5,885,829; and U.S. Patent Application Publication 20050079470. Most of those techniques involve the use of scaffolding materials that comprise mammalian cells such as dental pulp stem cells or mesenchymal stem cells, and/or bioactive ingredients such as bone morphogenic proteins (BMP). Techniques where cells are seeded onto the scaffolding material have the disadvantage of being difficult to prepare and store, since viable cells must be seeded, cultured and maintained on the scaffolding. Additionally, the source and yield of cells used in the regeneration of tissues can be inadequate.

There is thus a need for improvements in endodontic and root canal procedures, particularly restorative procedures using tissue engineering techniques. The present application addresses that need.

### SUMMARY

The present application is based on the discovery that diseased, traumatized or missing tooth pulp can be replaced with a composition comprising a bioactive ingredient that promotes angiogenic, odontogenic, fibrogenic or neurogenic development. Such a composition promotes angiogenic, odontogenic, fibrogenic or neurogenic development into the pulp chamber, preserving the vitality of the tooth.

The application is directed to a method of performing a dental, endodontic or root canal procedure on a mammalian tooth in need thereof. The method comprises exposing traumatized or diseased dental pulp tissue in the tooth pulp chamber and/or root canal; and capping or filling at least a portion of the tooth pulp chamber and/or root canal with a composition comprising a bioactive ingredient. The bioactive ingredient promotes angiogenic, odontogenic, fibrogenic, or neurogenic development. In these embodiments, the bioactive ingredient composition does not comprise a living cell during the capping or filling.

The application is also directed to a matrix, material or scaffold suitable for insertion into a tooth pulp chamber. The matrix, material or scaffold comprises a bioactive ingredient that promotes vascular tissue formation and/or nerve formation into the matrix, material or scaffold when the matrix, material or scaffold is inserted into the tooth pulp chamber. In these embodiments, the matrix, material or scaffold does not comprise a living cell.

Additionally, the application is directed to the use of the above matrix, material or scaffold in a dental, endodontic or root canal procedure.

The application is further directed to the use of the above matrix, material or scaffold for the manufacture of a medicament for a dental, endodontic or root canal procedure.

### BRIEF DESCRIPTION OF THE DRAWINGS

FIG. 1 is photographs of adult human teeth that underwent clinically equivalent root canal treatment. The endodontically treated root canal and pulp chamber were filled with collagen sponge without a bioactive ingredient (Panel a), or with basic fibroblast bioactive ingredient (bFGF) only (Panel b), or both bFGF and vascular endothelial bioactive ingredient (VEGF) (Panel c). The teeth were implanted subcutaneously in immunodeficient mice for 2 weeks to evaluate whether vascularization takes place in the endodontically treated root canal and pulp chamber. As opposed to the no bioactive ingredient treatment (collagen sponge only) (a), bFGF only (b) and VEGF and bFGF combined (c) both showed vascularization in the collagen sponge inserted in the root canal.

FIG. 2 shows micrographs of sections of adult human teeth treated as in FIG. 1. Panel A shows the root canal of a permanent human incisor with an implanted collagen sponge without a bioactive ingredient. There is a lack of any host tissue ingrowth from apical foramen following in vivo implantation in immunodeficient mice. Panel B shows a root canal of a permanent human incisor with a VEGF-loaded collagen sponge, showing the presence of vascularization (arrow), and host tissue ingrowth. The infiltrating host tissue is attached to the dentin. Panel C shows a root canal of a permanent human incisor with a bFGF-loaded collagen sponge, showing the presence of vascularization (arrow), and host tissue ingrowth. The infiltrating host tissue is attached to the dentin. Panel D shows a root canal of a permanent human incisor with a VEGF+bFGF-loaded collagen sponge showing the presence of vascularization (arrow), and host tissue ingrowth. The infiltrating host tissue is attached to the dentin.

FIG. 3 is a graph showing the release kinetics of TGFβ3 from PLGA microspheres in a 1% BSA solution. TGFβ3 was released in a sustained fashion up to 36 and 42 days from 50:50 or 75:25 co-polymer ratios of PLGA microspheres, respectively, as detected by ELISA. Initial burst-like release was observed for both co-polymer ratios, although the 50:50 PLA/PGA ratio yielded a more rapid release rate than the 75:25 PLA/PGA ratio did.

FIG. 4 is scanning electron micrographs showing the fabrication and degradation of PLGA microspheres. Panel A shows a representative SEM image of microspheres fabricated from poly-d-l-lactic-co-glycolic acid (PLGA) with 50:50 PLA/PGA ratio with encapsulated TGFβ3. Panel B shows a representative SEM image of the anticipated degradation of TGFβ3 encapsulating PLGA microspheres in PBS solution.

FIG. 5 is a graph showing the cumulative average release of BMP-7 from PLGA microspheres.

FIG. 6 is a graph showing the cumulative average release of NGF from PLGA microspheres.

### DETAILED DESCRIPTION

This application is based in part on the discovery that diseased, traumatized or missing tooth pulp can be replaced with a composition comprising a bioactive ingredient that promotes angiogenic, odontogenic, fibrogenic or neurogenic development. Such a composition promotes angiogenic, odontogenic, fibrogenic or neurogenic development into the pulp chamber, preserving the vitality of the tooth.

As used herein, the singular forms “a”, “an” and “the” are intended to include the plural forms as well, unless the context clearly indicates otherwise. Additionally, the use of “or” is intended to include “and/or”, unless the context clearly indicates otherwise.

The application is directed to a method of performing a dental, endodontic or root canal procedure on a mammalian tooth in need thereof. The method comprises exposing traumatized or diseased dental pulp tissue in the tooth pulp chamber and/or root canal; and capping or filling at least a portion of the tooth pulp chamber and/or root canal with a composition comprising a bioactive ingredient. The bioactive ingredient promotes angiogenic, odontogenic, fibrogenic or neurogenic development. In these embodiments, the composition does not comprise a living cell during the capping or filling. In some embodiments, the method further comprises removing traumatized or diseased dental pulp tissue from the tooth to create a tooth pulp chamber and/or root canal substantially devoid of traumatized or diseased tissue. In some embodiments, the composition comprises a matrix, material or scaffold.

As used herein, a dental procedure is any procedure involving teeth. For the present application, exemplary dental procedures are endodontic procedures, which involve tooth pulp. A root canal is a dental procedure where the entire tooth pulp and root canal tissue is removed and replaced with an inert material or a composition comprising a matrix, material or scaffold that restores living tissue in the pulp chamber. In some embodiments, the composition further comprises a bioactive ingredient.

The bioactive ingredient can be any compound that promotes angiogenic, odontogenic, fibrogenic or neurogenic development, including but not limited to cytokines or enzymes (e.g., tissue plasminogen activator or urokinase).

As used herein, a cytokine is a secreted protein or glycoprotein that mediates or regulates immunity, inflammation, or hematopoiesis. Cytokines are generally produced de novo in response to a stimulus. They bind to specific membrane receptors, which then signal the cell via second messengers to alter gene expression. Cytokines include lymphokines, monokines, chemokines, and interleukins.

The present application encompasses the use of any bioactive ingredient that promotes angiogenic, odontogenic, fibrogenic and/or neurogenic development into the matrix, material or scaffold. Non-limiting examples include vascular endothelial growth factor (VEGF), basic fibroblast growth factor (bFGF), platelet derived growth factor (PDGF), angiogenin, angiopoietin-1, del-1, follistatin, granulocyte colony-stimulating factor (G-CSF), hepatocyte growth factor/scatter factor (HGF/SF), interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-α (TGF-α), transforming growth factor-β (TGF-β), tumor necrosis factor-α (TNF-α), vascular endothelial growth factor (VEGF), matrix metalloproteinase (MMP), angiopoietin 1 (ang1), ang2, delta-like ligand 4 (DLL4), connective tissue growth factor (CTGF), bone morphogenic protein (BMP), nerve growth factor (NGF), brain derived nerve factor (BDNF), NT-4, NT-3 and epidermal growth factor.

In some embodiments of these methods, the bioactive ingredient is a VEGF, a bFGF, a BMP-7, an NGF or a CTGF. The role of naturally produced growth factors have been studied in tooth development (Klein et al., 2006), healing tooth extraction sockets (Lalani et al., 2005), and during orthodontic movement (Derringer and Linden, 1998). Angiogenic growth factors, particularly VEGF and bFGF, were found to be involved. It has been discovered that VEGF and bFGF in implanted collagen sponges are effective in restoring viable tissue in a pulp chamber and root canal when added to collagen sponges and inserted into the pulp chamber after a root canal procedure. See Example 1.

The present methods can be used on any mammal, including domestic animals such as cats, dogs, cows, sheep, goats, or pigs. In some embodiments, the mammal is a human.

In these methods, the bioactive ingredient can be from any mammalian species. In some embodiments, the bioactive ingredient is a human bioactive ingredient, particularly when the mammal being treated is a human. The bioactive ingredient may also be recombinant.

The composition in these methods can comprise more than one bioactive ingredient, for example two, three, four, or more bioactive ingredients. The additional bioactive ingredient can be any useful bioactive ingredient including an angiogenic growth factor or a morphogenic growth factor (including but not limited to BMPs) or any other bioactive ingredient. In some embodiments, the composition comprises a VEGF and a bFGF. In other embodiments, the composition comprises a BMP-7 and an NGF. In additional embodiments, the composition comprises a VEGF, a bFGF, a BMP-7 and an NGF.

In some embodiments where the composition comprises a VEGF and bFGF, the composition comprises about 0.001 ng to about 10,000 μg VEGF and about 0.001 ng to about 10,000 μg bFGF per gram of composition. In other embodiments, the composition comprises about 0.01 ng to about 1,000 μg VEGF and about 0.02 ng to about 2,000 μg bFGF per gram of composition. In additional embodiments, the composition comprises about 10 ng to about 200 ng VEGF and about 50 ng to about 500 ng bFGF. In further embodiments, the composition comprises about 33 ng VEGF and about 167 ng bFGF.

In some embodiments where the composition comprises BMP-7 and NGF, the composition comprises about 0.2 ng to 10,000 ng BMP-7 and about 0.2 ng to 500 ng NGF per gram of matrix, material or scaffold. In other embodiments, the composition comprises about 1 ng to 1000 ng BMP-7 and about 0.5 ng to 100 ng NGF. In additional embodiments, the bioactive ingredient composition comprises about 5 ng to 50 ng BMP-7 and about 1 ng to 10 ng NGF.

In some embodiments, the bioactive ingredient composition comprises an antibiotic. Exemplary antibiotics are penicillin V potassium, amoxicillin, augmentin, clindamycin or azithromycin.

In other embodiments, the bioactive ingredient composition comprises an analgesic. Exemplary analgesics are paracetamol, diclofenac, ketoprofen, aspirin, naproxen, indomethacin, ketorolac, ibuprofen, piroxicam, celecoxib, meloxicam, mefenemic acid, rofecoxib, nimesulide or a prostaglandin.

The bioactive ingredient composition can also comprise both an antibiotic and an analgesic.

As used herein, a “matrix” is an amorphous structure, e.g., a gel, in which the bioactive ingredients are suspended. A “material” is a fibrous composition, and a “scaffold” has tertiary structure, e.g., a columnar structure or a porous structure such as in a typical collagen sponge, e.g., with fairly uniform pores between about 250 and 400 μM, in which a bioactive ingredient solution permeates. The invention is not limited to any particular matrix, material or scaffold. Preferably, the matrix, material or scaffold is biodegradable.

In these methods, the bioactive ingredient can be combined with the matrix, material or scaffold by any means known in the art. In some embodiments, the bioactive ingredient is injected into the matrix, material or scaffold. In other embodiments, the bioactive ingredient is mixed into the matrix, material or scaffold. Further, the bioactive ingredient can be encapsulated in the matrix, material or scaffold, or chemically tethered to, or absorbed in, the matrix, material or scaffold, by methods known in the art.

The matrix, material or scaffold for these methods can be made from any compound known in the art as useful for these methods. In some embodiments, the matrix, material or scaffold comprises a natural polymer. Exemplary natural polymers are collagens and polysaccharides. In other embodiments, the matrix, material or scaffold comprises a synthetic polymer. Exemplary synthetic polymers are aliphatic polyesters of poly(α-hydroxy acid)s, polyethylene glycols, and chitosan. Additional synthetic polymers are polylactic acid (PLA), polyglycolic acid (PGA), and mixtures of PLA and PGA (PLGA). In some embodiments, the synthetic polymer is PLGA comprising about 50% PLA and 50% PGA.

In other embodiments, the matrix, material or scaffold comprises a collagen sponge or PLGA. In some embodiments, the collagen sponge or PLGA comprises a VEGF, a bFGF, a BMP-7 or an NGF.

One application of the instant methods is in a root canal procedure, where all pulp tissue is removed from the tooth. The matrix, material or scaffold would partially or completely replace current endodontic filing materials such as gutta-percha in those methods. The current methods do not exclude the combined use of the matrix, material or scaffold and current materials such as gutta-percha. Thus, in some embodiments, an inert material is also inserted into the pulp chamber, for example gutta-percha.

The replaced pulp could be due to any condition that a dental, endodontic or root canal procedure is prescribed to remedy. For example, the pulp tissue could have been infected with bacteria. Alternatively, the pulp tissue could have been damaged due to trauma, or there could be a defect in the pulp tissue.

The application is also directed to a matrix, material or scaffold suitable for insertion into a tooth pulp chamber. The matrix, material or scaffold comprises a bioactive ingredient that promotes angiogenic, odontogenic, fibrogenic or neurogenic development into the matrix, material or scaffold when the matrix, material or scaffold is inserted into the tooth pulp chamber, wherein the matrix, material or scaffold does not comprise a living cell. In some embodiments, the bioactive ingredient is a cytokine

Non-limiting examples of bioactive ingredients that promote vascular tissue formation include vascular endothelial growth factor (VEGF), basic fibroblast growth factor (bFGF), platelet derived growth factor (PDGF), angiogenin, angiopoietin-1, del-1, follistatin, granulocyte colony-stimulating factor (G-CSF), hepatocyte growth factor/scatter factor (HGF/SF), interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-α (TGF-α), transforming growth factor-β (TGF-β), tumor necrosis factor-α (TNF-α), vascular endothelial growth factor (VEGF), matrix metalloproteinase (MMP), angiopoietin 1 (ang1), ang2, delta-like ligand 4 (DLL4), connective tissue growth factor (CTGF), bone morphogenic protein (BMP), nerve growth factor (NGF), brain derived nerve factor (BDNF), NT-4, NT-3 or epidermal growth factor. The bioactive ingredient can be from any mammalian species. In some embodiments, the bioactive ingredient is a human bioactive ingredient. The bioactive ingredient can also be recombinant.

The matrix, material or scaffold in these methods can comprise more than one bioactive ingredient, for example two, three, four, or more bioactive ingredients. The additional bioactive ingredient can be any useful bioactive ingredient including an angiogenic growth factor or a morphogenic growth factor (including but not limited to BMPs) or any other bioactive ingredient. In some embodiments, the matrix, material or scaffold comprises a VEGF and a bFGF. In other embodiments, the matrix, material or scaffold comprises a BMP-7 and an NGF. In additional embodiments, the matrix, material or scaffold comprises a VEGF, a bFGF, a BMP-7 and an NGF.

In some embodiments where the matrix, material or scaffold comprises a VEGF and bFGF, the matrix, material or scaffold comprises about 0.001 ng to about 10,000 μg VEGF and about 0.001 ng to about 10,000 μg bFGF per gram of matrix, material or scaffold. In other embodiments, the matrix, material or scaffold comprises about 0.01 ng to about 1,000 μg VEGF and about 0.02 ng to about 2,000 μg bFGF per gram of matrix, material or scaffold. In additional embodiments, the matrix, material or scaffold comprises about 10 ng to about 200 ng VEGF and about 50 ng to about 500 ng bFGF. In further embodiments, the matrix, material or scaffold comprises about 33 ng VEGF and about 167 ng bFGF.

In some embodiments where the matrix, material or scaffold comprises BMP-7 and NGF, the matrix, material or scaffold comprises about 0.2 ng to 10,000 ng BMP-7 and about 0.2 ng to 500 ng NGF per gram of matrix, material or scaffold. In other embodiments, the matrix, material or scaffold comprises about 1 ng to 1000 ng BMP-7 and about 0.5 ng to 100 ng NGF. In additional embodiments, the composition comprises about 5 ng to 50 ng BMP-7 and about 1 ng to 10 ng NGF.

In some embodiments, the matrix material or scaffold comprises an antibiotic. Exemplary antibiotics are penicillin V potassium, amoxicillin, augmentin, clindamycin or azithromycin.

In other embodiments, the matrix, material or scaffold comprises an analgesic. Exemplary analgesics are paracetamol, diclofenac, ketoprofen, aspirin, naproxen, indomethacin, ketorolac, ibuprofen, piroxicam, celecoxib, meloxicam, mefenemic acid, rofecoxib, nimesulide or a prostaglandin.

The matrix, material or scaffold can also comprise an antibiotic and an analgesic.

In these methods, the bioactive ingredient can be combined with the matrix, material or scaffold by any means known in the art. In some embodiments, the bioactive ingredient is injected into the matrix, material or scaffold. In other embodiments, the bioactive ingredient is mixed into the matrix, material or scaffold. Further, the bioactive ingredient can be encapsulated in the matrix, material or scaffold, or tethered to, or absorbed in, the matrix, material or scaffold.

The matrix, material or scaffold for these methods can be made from any compound known in the art as useful for these methods. In some embodiments, the matrix, material or scaffold comprises a natural polymer. Exemplary natural polymers are collagens and polysaccharides. In other embodiments, the matrix, material or scaffold comprises a synthetic polymer. Exemplary synthetic polymers are aliphatic polyesters of poly(α-hydroxy acid)s, polyethylene glycols, and chitosan. Other exemplary synthetic polymers are polylactic acid (PLA), polyglycolic acid (PGA), or a mixture of PLA and PGA (PLGA). In some embodiments, the synthetic polymer is PLGA comprising about 50% PLA and 50% PGA.

In other embodiments, the matrix, material or scaffold comprises a collagen sponge or PLGA. In some aspects, the collagen sponge or PLGA comprises a VEGF, a bFGF, a BMP-7 or an NGF.

The application is additionally directed to the use of the above matrix, material or scaffold in a dental, endodontic or root canal procedure.

The application is further directed to the use of the above matrix, material or scaffold for the manufacture of a medicament for a dental, endodontic or root canal procedure.

Preferred embodiments are described in the following examples. Other embodiments within the scope of the claims herein will be apparent to one skilled in the art from consideration of the specification or practice of the invention as disclosed herein. It is intended that the specification, together with the example, be considered exemplary only, with the scope and spirit of the invention being indicated by the claims, which follow the examples.

### Example 1

Extracted human incisors were subjected to a root canal treatment. A collagen sponge, with or without bFGF and/or VEGF, was then implanted into the root canal. The incisors were then implanted subcutaneously in immunodeficient mice. The teeth were removed after two weeks and assessed for vascularization in the pulp chamber and root canal.

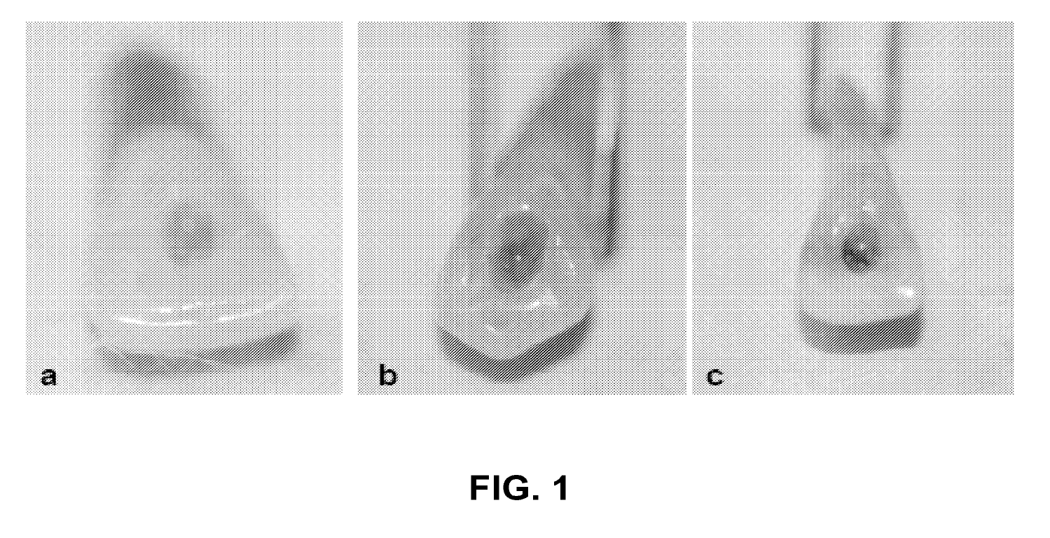

On visual inspection, the teeth treated with a collagen sponge without any bioactive ingredient had no apparent vascular development (FIG. 1a). However, teeth treated with a collagen sponge having bFGF or the combination of bFGF and VEGF showed vascularization in the collagen sponge inserted into the root canal (FIGS. 1b and 1c).

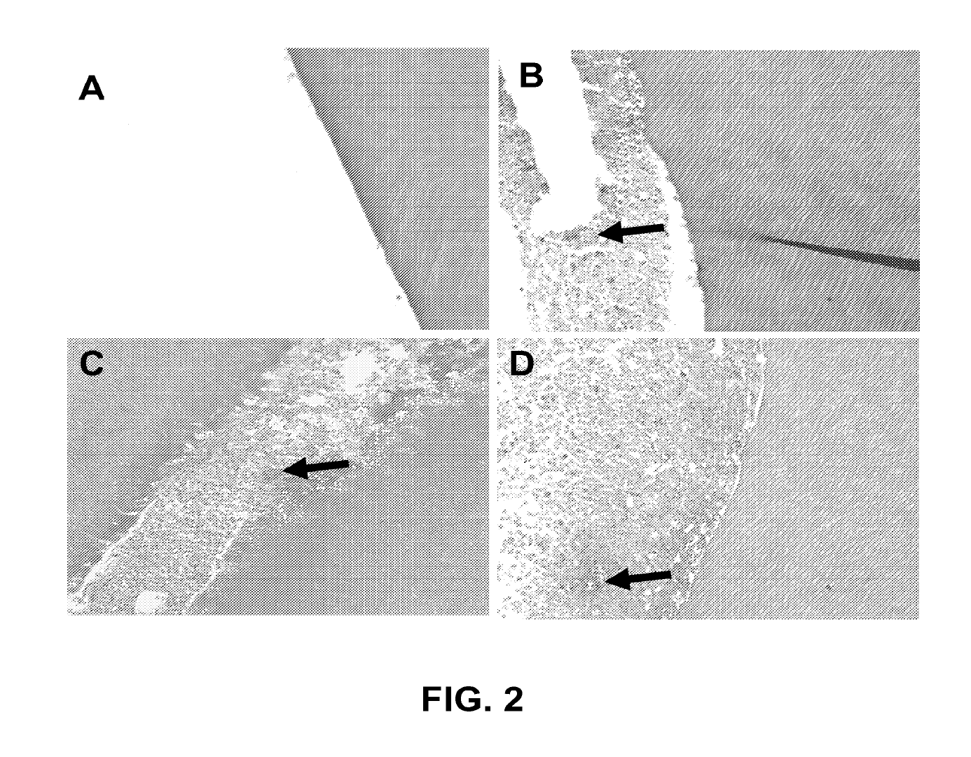

The root canal of the above-treated teeth were further evaluated microscopically. Teeth treated with a collagen sponge without any bioactive ingredient showed no tissue growth in the root canal (FIG. 2A) whereas teeth treated with a collagen sponge with either bFGF or VEGF or the combination of bFGF+VEGF showed vascularization and host tissue ingrowth (FIG. 2B-D). The infiltrating host tissue in those treatments was attached to the dentin.

### Example 2

### Regeneration of Dental Pulp: Odontogenesis and Neurogenesis

### Example Summary

The tooth is an organ that consists of enamel, dentin, pulp and cementum. During development, a number of key mediators are involved in the genesis of multiple tissues. Dental pulp is of particular importance because it remains the only dental tissue that is supplied by blood vessel in the adult. The dental pulp is populated by several cell populations including odontoblasts and nerves. The bodies of odontoblasts reside in the dental pulp and extend processes into the dentinal tubules. Nerve endings of pain fibers and sympathetic fibers are present in the dental pulp, and exert functions such as pain detection and regulation of blood vessels. Example 1 above demonstrates the genesis of blood vessels in the pulp chamber and root canal of endodontically treated human teeth. To further enable the regeneration of dental pulp, the encapsulation and controlled release of an odontogenic bioactive ingredient, bone morphogenic protein-7 (BMP-7), and a neurogenic bioactive ingredient, nerve bioactive ingredient (NGF), in a biocompatible microsphere, poly-d-l-lactic-co-glycolic acid (PLGA) is now demonstrated. PLGA was fabricated from 50:50 (PLA:PGA), and degraded slowly. BMP-7 and NGF were released gradually upon the degradation of PLGA microspheres over time. After four to six weeks, the release profiles of BMP-7 and NGF were determined by ELISA, and confirmed cumulative release concentration curves. These findings provide the proof of concept for applying BMP-7 and NGF in biocompatible microspheres for the regeneration of dental pulp in vivo.

### Introduction

About 40 million root canal procedures are performed in the U.S. each year. Root canal procedures are performed due to dental pulp infections or trauma. Dental pulp is the primary “live” portion of the adult tooth, and consists of blood vessels and blood-vessel-derived cells, nerve fibers and odontoblasts. Odontoblasts are responsible for elaborating dentin matrix, and extend their processes into dentinal tubules. Upon infection or trauma, dental pulp is removed in root canal therapy. Root canal therapy leads to a dead dental pulp, creating a “dead” tooth. Endodontically treated teeth become discolored and brittle, and need to be treated separately.

Example 1 above shows that empty pulp chambers and root canals of human teeth filled with collage sponges adsorbed with angiogenic bioactive ingredients generated vascularized pulp-like tissues in vivo. These findings are now expanded by enabling the regeneration of odontoblasts and nerve endings in the dental pulp post using the delivery of bioactive ingredients. The odontogenic and neurotrophic bioactive ingredients are used to induce the regeneration of odontoblasts and nerve endings by controlled release.

PLGA microencapsulation was chosen for the method of controlled release due to (1) its mode of degradation by hydrolysis and not by enzymes, (2) the simple manipulation of the kind of release profile (release duration can be extended or shortened, initial burst can be affected and tapered release can be created) needed by varying the polymer composition, (3) the potential to homogenize specific sizes from its large range of diameters by filtering and (4) its established demonstration of in vivo trials that create sustained delivery in a temporospatial manner.

### Materials and Methods

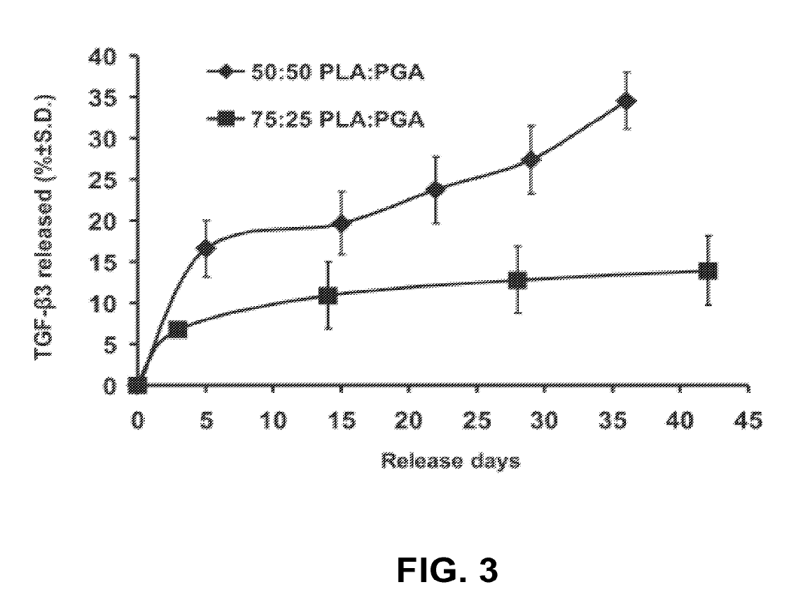

Preparation of PLGA microspheres and encapsulation of BMP-7 and NGF. Microspheres of poly-d-l-lactic-co-glycolic acid (PLGA, Sigma, St. Louis, Mo.) of 50:50 PLA/PGA ratio were chosen due to published findings on the cumulative release profile (Moioli et al., 2006; 2007a,b; Clark et al., 2007) (FIG. 3).

One hundred mL of 0.1% PVA was first prepared and put under continuous stirring for 30 minutes at 450 rpm before introducing any other constituent. The 50:50 ratio was prepared using the double emulsion technique ([water-in-oil]-in-water) (Moioli et al., 2006; 2007a,b; Clark et al., 2007). A total of 0.25 g of PLGA was fully dissolved in 1 mL of dichloromethane and emulsified (max vortex speed) with 2.5 μg of recombinant human BMP-7 or NGF diluted in 50 μL solution for 1 minute (water-in-oil). The primary emulsion was then vortexed with 2 mL of 1% polyvinyl alcohol (PVA, 30,000-70,000 MW) for 1 minute ([water-in-oil]-in-water). This mixture was then added to the stirring 0.1% PVA and stirred for 1 minute. A total of 100 mL of 2% isopropanol was added to the final emulsion and continuously stirred for 2 hours under the chemical hood to remove the solvent. PLGA microspheres containing the cytokines were isolated using filtration (2 μm filter), washed with distilled water and frozen in liquid nitrogen for 30 minutes and lyophilized for 48 hours. Freeze-dried PLGA microspheres were stored at −20° C. prior to use.

In vitro BMP-7 and NGF release kinetics. Both groups of odontogenic and neurotropic cytokine-encapsulated PLGA microspheres were distributed to 4 samples (n=4) each. Each group had 10 mg of the encapsulated cytokines in 1 mL of 1% BSA solution and was continuously agitated on shaker at 37° C. Data points were taken by collecting the entire amount of supernatants weekly for 4-6 weeks. 1 mL of 1% BSA solution was replaced after each collection. The amount of BMP-7 and NGF was quantitatively measured by using the BMP-7 ELISA kit and NGF ELISA kit for each sample.

### Results

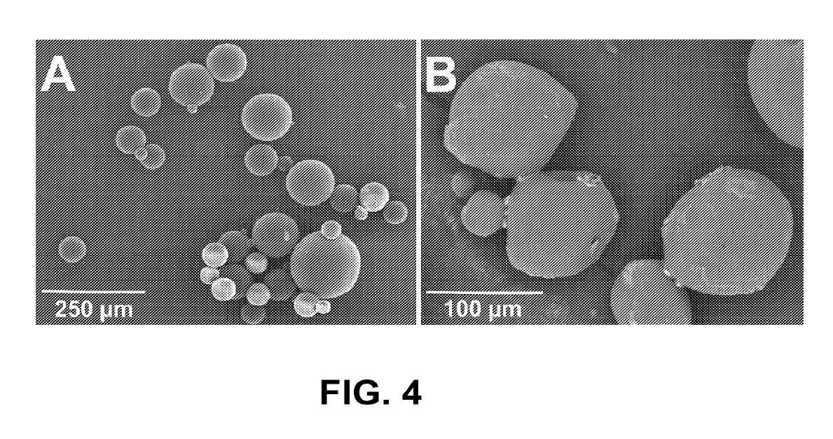

BMP-7 and NGF encapsulated in PLGA microspheres. BMP-7 and NGF encapsulating PLGA microspheres prepared by double-emulsion solvent-extraction technique produces a spherical shape and smooth surface that degrades over time, a characteristic of all microspheres. FIG. 4A is a scanning electron microscopy (SEM) image of TGFβ3 encapsulated microspheres (Moioli et al., 2006). After residing in 1% BSA for 4 days, PLGA microspheres began to show morphological changes and surface degradation (FIG. 4B).

BMP-7 and NGF release kinetics. BMP-7 and NGF microspheres were released up to 30-44 days in vitro with the 50:50 ratio of PLA/PGA. A burst-like release was found during the first week and showed similar release profiles compared to previously published results for TGFβ3 controlled release (FIG. 3). Both release profiles showed that 50:50 PLGA could encapsulate BMP-7 and NGF and have similar degradation rates as other previous encapsulated bioactive ingredients.

### Discussion

The present findings of sustained release of BMP-7 and NGF in PLGA microspheres should enable the regeneration of odontoblasts and nerve endings in root canal-treated human teeth. Long-term delivery of these bioactive ingredients via a controlled release approach may regulate cell recruitment, proliferation, and differentiation in an orderly fashion.

BMP-7 induces cellular proliferation, and expression of Msx-1, Msx-2, and BMP-4 in molar-forming mesenchyme after 24 hours in developing mice (Wang et al., 2000). This previous work provides the rationale for the use of BMP-7 in the induction of odontoblasts, although the approach in Wang et al. (2000) is to investigate the involvement of BMP-7 in tooth development. NGF mediates cell growth and differentiation of neuronal cells (Christensen et al., 1993). NGF expression of the human dental papilla was found to be transient and present in the condensing ecto-mesenchymal cells of the dental papilla in the early cap stage tooth germ (Christensen et al., 1993).

Sustained release enables prolonged delivery of the bioactive ingredient in contrast to diffusion, inactivation, and loss of bioactivity associated with bioactive ingredient injection. The release profiles of BMP-7 and NGF from PLGA microspheres suggest that the sustained release rates and initial bursts of BMP-7 and NGF from PLGA microspheres can be readily tailored to specific degradation requirements in the simulation of the bioactive ingredient delivery in vivo by further modifying the PLA/PGA ratio, if needed. The methyl group in PLA is responsible for its hydrophobic and slow degradation. PGA is crystalline and increases degradation times. Therefore, different ratios of PGA and PLA are likely necessary for various applications in wound healing and tissue engineering to accommodate specific bioactive ingredient release rates.

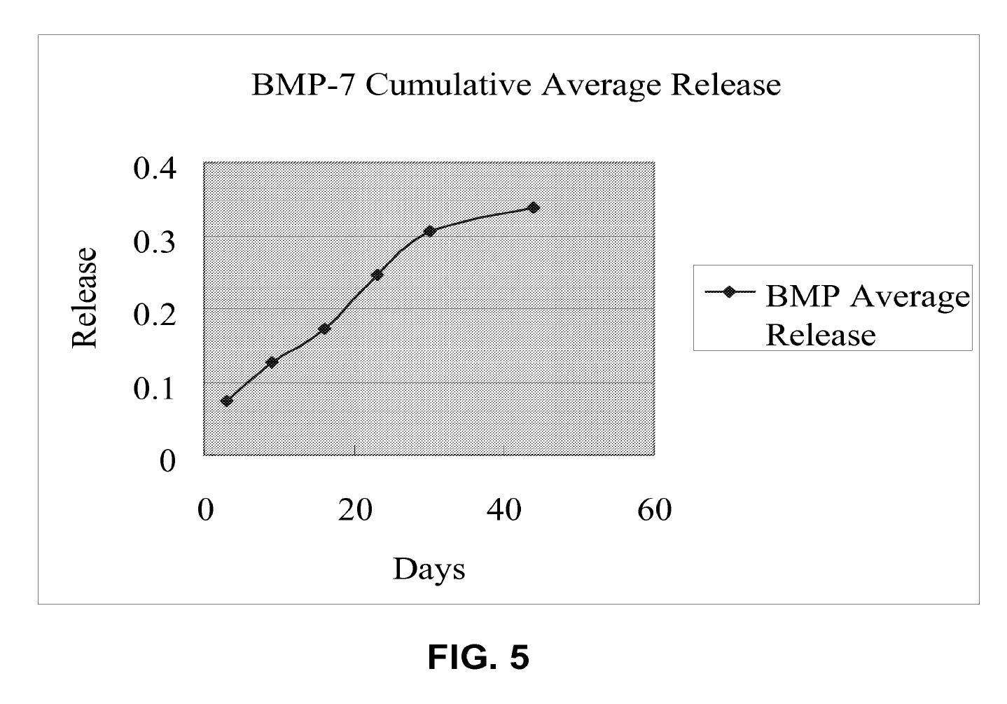

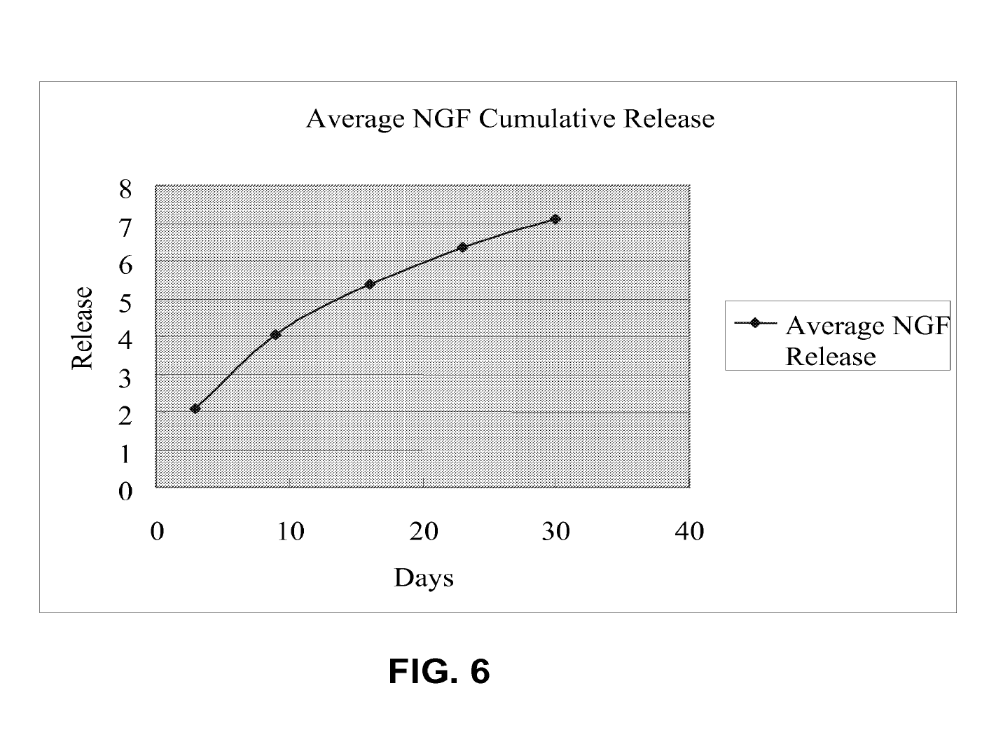

Different concentrations of bioactive ingredients can be delivered with similar release profiles from microspheres, but with corresponding doses (Clark et al., 2007). BMP-7 shows a substantially smaller release concentration relative to NGF, which may be attributed to specific bioactive ingredient-polymer interactions. However, BMP-7 in its natural environment requires a faster and larger initial burst to initiate all other cellular responses at the beginning of development. The release profile shown in Table 1 and FIG. 5 does not depict an ideal curve as NGF does in Table 2 and FIG. 6. Both BMP-7 and NGF release rates from PLGA microspheres appear to be consistent with previous demonstration of hydrolysis of PLGA microspheres in an aqueous environment (Moioli et al., 2006; 2007a,b; Clark et al., 2007).

TABLE 1
Release of BMP-7 over time - ELISA data

| BMP-7 | Sam- | Sam- | Sam- | Sam- | BMP-7 | Cumulative |
|---|---|---|---|---|---|---|
| Release | ple | ple | ple | ple | Released/wk | Release |
| Time (days) | A | B | C | D | (ng/mL) | (ng/mL) |
| 0 | 0 | 0 | 0 | 0 | 0 | 0 |
| 3 | 0.072 | 0.073 | 0.074 | 0.077 | 0.074 | 0.074 |
| 9 | 0.05 | 0.055 | 0.058 | 0.05 | 0.05325 | 0.12725 |
| 16 | 0.042 | 0.055 | 0.042 | 0.039 | 0.0445 | 0.17175 |
| 23 | 0.071 | 0.083 | 0.074 | 0.073 | 0.07525 | 0.247 |
| 30 | 0.057 | 0.08 | 0.055 | 0.045 | 0.05925 | 0.30625 |
| 44 | 0.032 | 0.029 | 0.033 | 0.03 | 0.031 | 0.33725 |

TABLE 2
NGF ELISA data showing weekly and cumulative
release from PLGA microspheres

| NGF | Sam- | Sam- | Sam- | Sam- | NGF | Cumulative |
|---|---|---|---|---|---|---|
| Release | ple | ple | ple | ple | Released/wk | Release |
| Time (days) | A | B | C | D | (ng/mL) | (ng/mL) |
| 0 | 0 | 0 | 0 | 0 | 0 | 0 |
| 3 | 1.921 | 2.174 | 2.907 | 1.374 | 2.094 | 2.094 |
| 9 | 2.348 | 1.809 | 2.972 | 0.718 | 1.96175 | 4.05575 |
| 16 | 0.964 | 1.56 | 2.332 | 0.39 | 1.3115 | 5.36725 |
| 23 | 0.618 | 1.284 | 1.332 | 0.703 | 0.98425 | 6.3515 |
| 30 | 0.47 | 1.112 | 0.864 | 0.585 | 0.75775 | 7.10925 |

### REFERENCES

Alhadlaq A and Mao J J. J. Dent. Res. 2003 82:951-956.
Brennan M T, Kent M L, Fox P C, Norton H J, Lockhart PB. The impact of oral disease and nonsurgical treatment on bacteremia in children. J. Am. Dent. Assoc. 2007 January; 138(1): 80-85.
Christensen, L. R., Møllgård, K., Kjer, I. and Janas, M. S. Immunocytochemical demonstration of nerve bioactive ingredient receptor (NGF-R) in developing human fetal teeth. Anatomy and Embryology Vol 188, Number 3, 247, 1993
Derringer K A et al. Eur. J. Orthodontics 1998 20:357-367.
Edwards P C and J M Mason Head & Face Medicine 2006 2:16.
Fong H K et al. J. Dent. Education 2005 69:555-570.
Goldberg M and Smith A J. Crit. Rev. Oral Biol. Med. 2004 15:13-27.
Hong L and Mao J J. J. Dent. Res. 2004 83:751-756.
Klein O et al. p. 13 in COST Action B23 ORAL FACIAL DEVELOPMENT AND REGENERATION, Joint Meeting of the Working Group 1-4 and the Management Committee, BIOACTIVE INGREDIENTS IN CRANIOFACIAL DEVELOPMENT AND REPAIR, 4-7 May 2006.
Lalani Z et al. J. Oral Maxillofacial Surg. 2005 63:1500-1508.
Lovschall H et al. Adv. Dent. Res. 2001 15:108-112.
Mao J J, Giannobile W V, Helms J A, Hollister S J, Krebsbach P H, Longaker M T, Shi S. Craniofacial tissue engineering by stem cells. J. Dent. Res. 2006 November; 85(11):966-79.
Mathieu S et al. p. 79 in COST Action B23 ORAL FACIAL DEVELOPMENT AND REGENERATION, Joint Meeting of the Working Group 1-4 and the Management Committee, BIOACTIVE INGREDIENTS IN CRANIOFACIAL DEVELOPMENT AND REPAIR, 4-7 May 2006.
Moioli, E. K., Hong, L., Guardado, J., Clark, P., and Mao, J. J. Sustained release of TGFβ from PLGA microspheres and its effect on early osteogenic differentiation of human mesenchymal stem cells. Tissue Engineering Vol. 12, Number 3, 537, 2006
Murray P E et al. Crit. Rev. Oral Biol. Med. 2002 13:509-520.
Murray P E et al. J. Endod. 2007 33:377-390.
Nakashima M and Akamine A. J. Endod. 2005 31:711-718.
Nakashima M and Reddi A H. Nat. Biotechnol. 2003 21:1025-1032.
Salvi G E, Siegrist Guldener B E, Amstad T, Joss A, Lang N P. Clinical evaluation of root filled teeth restored with or without post-and-core systems in a specialist practice setting. Int Endod J. 2007 March; 40(3):209-15.
Shay K. Infectious complications of dental and periodontal diseases in the elderly population. Clin Infect Dis. 2002 May 1; 34(9):1215-23.
Stosich M S and Mao J J. Plast. Reconstr. Surg. 2007 119:71-83.
Wang, Y. H., Rutherford, B., Upholt, W. B. and Mina, M. Effects of BMP-7 on mouse tooth mesenchyme and chick mandibular mesenchyme. Developmental Dynamics Vol 216, Issue 4/5, 320, 2000.
Young C. S. et al. J. Dent. Res. 2002 81:695-700.
U.S. Pat. No. 5,885,829.
U.S. Patent Application Publication No. 20050079470.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1-83. (canceled)

84. A method of performing a dental, endodontic or root canal procedure on a mammalian tooth in need thereof, the method comprising:
exposing traumatized or diseased dental pulp tissue in the tooth pulp chamber or root canal; and
capping or filling at least a portion of the tooth pulp chamber or root canal with a composition comprising
(i) at least one bioactive agent; and
(ii) a matrix, material or scaffold;
wherein
the at least one bioactive agent promotes angiogenic, odontogenic, fibrogenic, or neurogenic development; and
the composition does not comprise a living cell during the capping or filling.

85. The method of claim 84, comprising at least one of:
removing traumatized or diseased dental pulp tissue from the tooth to create a tooth pulp chamber or root canal substantially devoid of traumatized or diseased tissue;
removing substantially all dental pulp tissue from the tooth; or
filling at least a portion of the tooth pulp chamber with an inert material.

86. The method of claim 84, wherein the at least one bioactive agent is a cytokine.

87. The method claim 84, wherein the at least one bioactive agent is selected from the group consisting of: a vascular endothelial growth factor (VEGF); a basic fibroblast growth factor (bFGF); a platelet derived growth factor (PDGF); an angiogenin; an angiopoietin-1; a del-1; a follistatin; a granulocyte colony-stimulating factor (G-CSF); a hepatocyte growth factor/scatter factor (HGF/SF); an interleukin-8 (IL-8); a leptin; a midkine; a placental growth factor; a platelet-derived endothelial cell growth factor (PD-ECGF); a platelet-derived growth factor-BB (PDGF-BB); a pleiotrophin (PTN); a progranulin; a proliferin; a transforming growth factor-α (TGF-α); a transforming growth factor-β (TGF-β); a tumor necrosis factor-α (TNF-α); a vascular endothelial growth factor (VEGF); a matrix metalloproteinase (MMP); an angiopoietin 1 (ang1); an ang2; a delta-like ligand 4 (DLL4); a connective tissue growth factor (CTGF); a bone morphogenic protein (BMP); a nerve growth factor (NGF); a brain derived nerve factor (BDNF); an NT-4; an NT-3; and an epidermal growth factor (EGF).

88. The method of claim 87, wherein the at least one bioactive agent is selected from the group consisting of: a VEGF; a bFGF; a BMP-7; an NGF; and a CTGF.

89. The method claim 84, wherein the composition comprises at least two bioactive agents, a first bioactive agent comprising a VEGF and a second bioactive agent comprising a bFGF.

90. The method of claim 89, wherein the composition comprises
(i) about 0.001 ng to about 10,000 μg VEGF per gram of composition; and about 0.001 ng to about 10,000 μg bFGF per gram of composition;
(ii) about 0.01 ng to about 1,000 μg VEGF and about 0.02 ng to about 2,000 μg bFGF per gram of composition;
(iii) about 10 ng to about 200 ng VEGF and about 50 ng to about 500 ng bFGF; or
(iv) about 33 ng VEGF and about 167 ng bFGF.

91. The method claim 84, wherein the composition comprises at least two bioactive agents, a first bioactive agent comprising a BMP-7 and a second bioactive agent comprising an NGF.

92. The method of claim 91, wherein the composition comprises
(i) about 0.2 ng to 10,000 ng BMP-7 and about 0.2 ng to 500 ng NGF per gram of composition;
(ii) about 1 ng to 1000 ng BMP-7 and about 0.5 ng to 100 ng NGF; or
(iii) about 5 ng to 50 ng BMP-7 and about 1 ng to 10 ng NGF.

93. The method claim 84, wherein the composition comprises at least four bioactive agents; a first bioactive agent comprising a VEGF; a second bioactive agent comprising a bFGF; a third bioactive agent comprising a BMP-7; and a fourth bioactive agent comprising an NGF.

94. The method of claim 84, wherein the bioactive ingredient is a human bioactive ingredient or a recombinant bioactive ingredient.

95. The method of claim 84, wherein the composition comprises:
at least one antibiotic;
at least one analgesic; or
at least one antibiotic and at least one analgesic.

96. The method of claim 84, wherein the at least one bioactive agent is injected into, mixed into, encapsulated in, tethered to, or absorbed in the matrix, material, or scaffold.

97. The method of claim 84, wherein the matrix, material or scaffold comprises:
a natural polymer selected from the group consisting of collagen, gelatin, a polysaccharide, hydroxyapatite (HA), and a polyhydroxyalkanoate; or
a synthetic polymer selected from the group consisting of an aliphatic polyester of a poly(α-hydroxy acid), a polyethylene glycol, and chitosan.

98. The method of claim 97, wherein the synthetic polymer is polylactic acid (PLA), polyglycolic acid (PGA), or a mixture of PLA and PGA (PLGA).

99. The method of claim 97, wherein the synthetic polymer is PLGA comprising about 50% PLA and about 50% PGA.

100. The method of claim 86, wherein the matrix, material or scaffold comprises a collagen sponge or PLGA.

101. The method of claim 85, wherein the matrix, material or scaffold is biodegradable.

102. The method of claim 84, wherein the tooth is in a human.

103. A composition suitable for insertion into a tooth pulp chamber, the composition comprising
(i) at least one bioactive agent; and
(ii) a matrix, material or scaffold;
wherein
the at least one bioactive agent promotes angiogenic, odontogenic, fibrogenic, or neurogenic development when the composition is inserted into a tooth; and
the composition does not comprise a living cell.
